(19) United States
(12) Patent Application Publication
(10) Pub. No.: US 2007/0231275 A1
(43) Pub. Date: Oct. 4, 2007

(54) Method for regenerating tooth germ

(76) Inventor: Minoru Ueda, Aichi (JP)

Correspondence Address: ANTONELLI, TERRY, STOUT & KRAUS, LLP, 1300 NORTH SEVENTEENTH STREET, SUITE 1800, ARLINGTON, VA, 22209-3873, US

(21) Appl. No.: 10/555,790

(22) PCT Filed: Feb. 27, 2004
(86) PCT No.: PCT/JP04/02409

§ 371 Date: Dec. 13, 2006

(30) Foreign Application Priority Data
May 7, 2003 (JP) 2003-138910

(51) Int. Cl. A61K 35/32; A61K 8/96

(52) U.S. Cl. 424/50; 424/93.7

(57) ABSTRACT

It is an object of the present invention to provide a method for regenerating tooth germ, and more specifically, to provide a method for regenerating tooth germ with a size sufficient for enabling the treatment of patients who have lost teeth or have had teeth damaged by dental diseases such as pyorrhea alveolaris or dental caries. The present invention provides a method for regenerating tooth germ, which comprises culturing tooth germ cells in the presence of a physiologically active substance.

### TECHNICAL FIELD

The present invention relates to a method for regenerating tooth germ. More specifically, the present invention relates to a method for regenerating tooth germ by culturing tooth germ cells in the presence of a physiologically active substance. The present invention also relates to a method for treating patients with dental diseases using tooth germ regenerated by the above method.

### BACKGROUND ART

The modern society is an aging society, and it is predicted that elderly people over age 65 will make up approximately 20% of the total population in Japan in several years. A majority of such elderly people have lost a part or all of their teeth for various reasons, and many of them use artificial teeth (what are called “false teeth”). The conventional artificial tooth has been problematic, not only in that it requires to be put on and taken off and causes an uncomfortable feeling when it is attached, but also in that the use thereof imposes psychological pressure upon patients, giving an impression as a symbol of aging. Thus, it is recognized that patients are generally reluctant to use such an artificial tooth. In addition, it has been known that when full dentures are attached because all teeth were lost, chewing ability becomes approximately one-fifth of that of natural teeth. It is not negligible that diet, which may be a pleasure for many elderly people, often causes pain after the loss of teeth. Moreover, it has been clarified that the masticatory stimulus upon the brain has effects of preventing dementia, and thus, that a decrease in chewing ability promotes dementia.

Under these circumstances, a dental implant has been developed and applied to clinical sites in recent years. Application of such a dental implant has achieved the fixing of artificial teeth, allowed for easy maintenance, and improved chewing ability. However, it has not yet been satisfactory in terms of esthetics or comfortable fitting. Moreover, it cannot be said that implant dentures have widely been used, for the reasons that it requires surgery; that it requires a certain amount of bone, and thus, the use of the dental implant is restricted depending on the general status of a patient; and that it has a high cost, and further reliable medical institutions are also limited. Consequently, although there are many patients who use artificial teeth and are not satisfied with them, only a very limited number of patients use implant dentures.

Transplantation of teeth by allotransplantation has been reported. However, it is difficult to remove and maintain transplantable healthy teeth, and such type of tooth transplantation involves the risk of infectious diseases. Thus, allotransplantation has not yet become a common treatment. There are many patients who do not venture to try dental implants although they are not satisfied with artificial teeth, or who have difficulty in undergoing a treatment with implant dentures due to their individual conditions.

To date, regarding studies of dental regeneration, regeneration of periodontal tissues have become a focus of attention, and related studies have been undertaken mainly for regeneration of bones and periodontal membranes. As a result of these studies, the GTR method (Guided Tissue Regeneration method) has been developed. The GTR method involves preventing epidermic cells from entering the surface of a tooth root, using a membrane such as Millipore Filter (product name; Millipore Corp.), so as to form a space necessary for the growth of periodontal cells (Nyman et al., J. Clin. Periodontol., 9, 290 (1982)). The GTP method intends to regenerate alveolar bones and periodontal membranes around teeth affected by periodontal disease. This method yields good results in the case of mild periodontal diseases. Moreover, in recent years, a protein capable of regenerating a periodontal membrane has been developed and practically used. However, the GTR method cannot be applied when there is a high degree of absorption regarding alveolar bones, which causes the loss of teeth. Further, it cannot repair the collapse of teeth caused by dental caries.

In order to fundamentally solve the aforementioned problems, a method for regenerating tooth germ itself has been proposed and studied (C. S. Young et al J. Dent. Res. 81 (10), 2002). However, only small tissues have been formed by this method, and tissues that were large enough to be used in practical treatment have not been formed.

### DISCLOSURE OF THE INVENTION

It is an object of the present invention to solve the aforementioned problems of the prior art techniques. In other words, it is an object of the present invention to provide a method for regenerating tooth germ, and more specifically, to provide a method for regenerating tooth germ with a size sufficient for enabling the treatment of patients who have lost teeth or have had teeth damaged by dental diseases such as pyorrhea alveolaris or dental caries. Moreover, it is another object of the present invention to provide a method for treating patients who have lost teeth or have had teeth damaged, using regenerated tooth germ.

As a result of intensive studies directed towards achieving the aforementioned objects, the present inventors have found that the induction of differentiation and the growth of tooth germ cells can be promoted by culturing the tooth germ cells in the presence of a physiologically active substance, and further that the survival rate of the cultured tooth germ cells can also be improved, thereby completing the present invention.

That is to say, the present invention provides a method for regenerating tooth germ, which comprises culturing tooth germ cells in the presence of a physiologically active substance.

Preferably, tooth germ cells are cultured on a carrier in the presence of a physiologically active substance.

Preferably, there is provided a method for regenerating tooth germ, which comprises: culturing tooth germ cells in the presence of a physiologically active substance; transplanting the cultured tooth germ cells into a transplanted animal; and regenerating tooth germ in the body of said transplanted animal.

More preferably, there is provided a method for regenerating tooth germ, which comprises: culturing tooth germ cells on a carrier in the presence of a physiologically active substance; transplanting the cultured tooth germ cells together with said carrier into a transplanted animal; and regenerating tooth germ in the body of said transplanted animal.

Preferably, odontoblasts, ameloblasts, pulp or dental papilla cells, tooth sac cells, or precursor cells thereof, are used as tooth germ cells.

Preferably, a growth factor is used as a physiologically active substance. More preferably, fibroblast growth factor (FGF), transforming growth factor (TGF), or a factor belonging to these families, is used as a physiologically active substance. Particularly preferably, GDF-5 or FGF-4 is used as a physiologically active substance.

The present invention further provides a tooth germ regenerated by the aforementioned method for regenerating tooth germ according to the present invention.

The present invention further provides a method for treating patients with dental diseases, which comprises transplanting the tooth germ regenerated by any of the aforementioned methods into the jawbone of a patient who has lost his or her own tooth germ or has had his or her own tooth damaged.

The present invention further provides an implant used for live organ transplant, which is obtained by culturing tooth germ cells in the presence of a physiologically active substance.

The present invention further provides an implant used for live organ transplant, which is obtained by: culturing tooth germ cells in the presence of a physiologically active substance; transplanting the cultured tooth germ cells into a transplanted animal; and regenerating tooth germ in the body of said transplanted animal.

The present invention further provides an implant used for live organ transplant, which is obtained by: culturing tooth germ cells on a carrier in the presence of a physiologically active substance; transplanting the cultured tooth germ cells together with said carrier into a transplanted animal; and regenerating tooth germ in the body of said transplanted animal.

### BRIEF DESCRIPTION OF THE DRAWINGS

FIG. 1 shows the number of cells on the 5^{th} day after addition of GDF-5;

FIG. 2 shows alkaline phosphatase activity on the 5^{th} day after addition of GDF-5;

FIG. 3 shows regenerated tooth germ at 15 weeks after transplantation of the tooth germ cells cultured in the presence of GDF;

FIG. 4 shows a histology of tissues (stained with hematoxylin and eosin), which were extirpated at 15 weeks after transplantation; and

FIG. 5 shows alkaline phosphatase activity on the 4^{th} day after addition of FGF-4.

### BEST MODE FOR CARRYING OUT THE INVENTION

The embodiments of the present invention will be described in detail below.

The method of the present invention for regenerating tooth germ is characterized in that tooth germ cells are cultured in the presence of a physiologically active substance.

The type of a tooth germ cell used in the present invention is not particularly limited, as long as the cell constitutes tooth germ or can differentiate into a tooth germ cell. Examples of such cells may include odontoblasts, ameloblasts, pulp or dental papilla cells, tooth sac cells, or precursor cells thereof. These cells may be cultured as single cells consisting of one type of cell, or may also be cultured as cell mixtures consisting of two or more types of cells.

Tooth germ cells can be collected from the lower jawbone of a mammal (for example, a human, a swine, etc.). An impacted tooth is aseptically excised, and it is then conserved in a suitable preservation solution such as a Hanks balanced salt solution (HBSS). A calcified portion is removed from the tooth, and the residual tissues are fragmented using a knife. The fragmented tissues are then washed with an HBSS solution or the like. Subsequently, the tissues are preferably subjected to an enzyme treatment with collagenase and dispase. After completion of such an enzyme treatment, cells can be recovered by pipetting and centrifugation.

The tooth germ regenerated by the method of the present invention is transplanted to a dental patient (that is, a patient who suffers from the loss of a tooth or a damaged tooth), and thus, it is used in the treatment of such a patient. In this case, from the viewpoint of biocompatibility associated with transplantation, tooth germ cells used in regeneration are preferably the patient's own tooth germ cells. Cells constituting tooth germ or cells differentiating into tooth germ can also be collected from wisdom teeth.

It has been known that a tooth is formed in 5 stages ranging from generation to maturation. The first stage is called the initiation stage, when epithelial tissues and mesenchymal tissues are induced to the basement membrane. The second stage is called the bud stage, when an enamel organ is generated. The third stage is called the cap stage, when dental papilla is generated and tooth germ is then generated. The fourth stage is called the bell stage, when both differentiation of the tooth germ into cells forming dental enamel and differentiation of the dental papilla into cells forming dentin and dental pulp are initiated. The fifth stage is called the maturation stage, when cells are differentiated into tissues constituting the tooth, such as dental enamel, dentin, and dental pulp. In the present invention, cells in a preferred stage selected from the aforementioned stages are collected and used. In a case where no tooth germ exists, dental pulp or periodontal membrane is excised from a tooth root, and cells are then separated and collected therefrom. It is to be noted that excision of dental pulp from a tooth can be carried out by the method described in About I et al., Experimental Cell Research. 258. 33-41, 2000.

In the present invention, the term “regeneration of tooth germ” is used to mean regeneration of tooth germ obtained from the second stage onwards, from among the aforementioned 5 stages.

In the present invention, tooth germ cells are cultured in the presence of a physiologically active substance. The type of a physiologically active substance used herein is not particularly limited, as long as it is able to promote the differentiation induction or growth of tooth germ cells, or is able to improve the survival rate of the cultured tooth germ cells. Examples of such a physiologically active substance may include various types of growth factors, low molecular weight compounds, and inorganic salts (for example, calcium phosphate, etc.).

Examples of a growth factor that can be used in the present invention may include fibroblast growth factor (FGF), hepatocyte growth factor (HGF), hormone, cytokine, glia-derived neurotrophic factor (GDNF), bone morphogenetic protein (BMP), transforming growth factor (TGF) (for example, TGF-α, TGF-β, TGF-γ, etc.), epidermal growth factor (EGF), insulin, insulin-like growth factor (IGF), nerve growth factor (NGF), platelet-derived growth factor (PDGF), colony-stimulating factor (CSF), vascular endothelial growth factor (VEGF), erythropoietin, transferrin, interleukin, and factors belonging to these families.

All of the aforementioned factors have been known. For example, products that are commercially available from Gibco or Upstate Biotechnology can be used. In addition, growth factors, which had not yet been known at the time of filing of the present application, can also be used as with the aforementioned growth factors, as long as they are able to promote the differentiation induction or growth of tooth germ cells or are able to improve the survival rate of the cultured tooth germ cells.

In the present invention, among the aforementioned growth factors, GDF-5 (Growth Differentiation Factor-5) and GDF-6 (Growth Differentiation Factor-6) belonging to the transforming growth factor (TGF) family, and FGF-4 belonging to the fibroblast growth factor (FGF) family, are particularly preferably used.

The concentration of a physiologically active substance to be added is different depending on the tooth germ cells which are used and the physiologically active substance which is used. The concentration is between 0.0001 and 10 μg/ml, and preferably between 0.001 and 1 μg/ml in a medium used. Such a physiologically active substance can be added not only when the culture is initiated, but also regularly, or when the medium is exchanged with a fresh one, or when the subculture is initiated. Moreover, it is also possible that a physiologically active substance-supported carrier is used, and that the culture be then carried out while the physiologically active substance is slowly released.

The cells can be cultured, using a common medium containing serum that is used in the culture of animal cells, under common conditions for culturing animal cells (for example, at a temperature between room temperature and 37° C., in a 5% CO₂ incubator). In addition, it is also possible to add stimulation such as mechanical stimulation during the culture.

When tooth germ cells are cultured in the presence of a physiologically active substance in the present invention, the tooth germ cells may be cultured on a carrier, or may be cultured with no carriers. However, the tooth germ cells are preferably cultured on a carrier. The use of a carrier is useful for forming tooth germ tissues from the cells. It is preferable to use a carrier, which endures a period of time necessary for formation of tooth germ, and which is then rapidly absorbed into a body. That is to say, it is preferable to use a carrier, which has a suitable speed and properties of being absorbed into a living body such as the greater momentum attached to the stomach, or the jawbone, and the material of which has high affinity to the cells.

The material of such a carrier is not particularly limited, as long as it satisfies the aforementioned properties. Examples of such a material may include polyglycolic acid (PGA), poly(DL-lactide-co-glycolide) (PLGA), poly-ε-caprolactone, and collagen. Also, natural materials such as a dentin including a protein may be used.

PGA is commercially available from Albany International Research Co. and other companies. PLGA is commercially available from Sigma. In the case of PGA, since this compound is rapidly absorbed, it is also possible to coat the surface thereof with poly(DL-lactide) (PLLA), so as to retard the absorption period. Moreover, when synthetic materials such as PGA, PLGA, or poly-ε-caprolactone are used, the surfaces of these compounds are coated with a collagen solution or the like and then used, so as to enhance the adhesiveness of the cells.

Examples of a possible form as a carrier may include a mesh form, a sponge form, and a gel form. In the case of a carrier having a gel form, cells come into contact with one another more easily than in the case of a carrier having a mesh or sponge form. Accordingly, among others, a carrier having a gel form is useful for the culture of tooth germ cells.

There is preferably used a carrier that is processed into a form, which facilitates transplantation of the cells. Such a carrier preferably has a platy, spherical, or hollow form, one end of which is open, so that blood circulation can easily be introduced from surrounding portions.

It is preferable to produce a carrier with a form that is suitable for purpose. Thus, a form of interest is produced from resin, and then, a mold is obtained using an impression material. Thereafter, the mold of resin is taken out, and a synthetic material constituting a carrier is poured therein, so as to replicate the form of interest.

In the method of the present invention, tooth germ cells may be cultured in the presence of a physiologically active substance, and that the cultured cells may be then transplanted into a transplanted animal, so as to regenerate tooth germ in the body of the transplanted animal. Alternatively, it may also be possible that the above cultured cells be directly transplanted into the jawbone of a patient. Preferably, a carrier used in the culture of tooth germ cells is also transplanted into the body of the transplanted animal, together with the tooth germ cells.

The type of a transplanted animal is not particularly limited, but it is preferably a mammal. Examples of a mammal used herein may include rodents such as a rat (e.g. a nude rat), rabbit, or mouse. As a site into which tooth germ cells are transplanted, a site to which factors necessary for formation of tooth germ can easily be supplied is preferable. More specifically, a site having a high blood flow, such as the greater momentum attached to the stomach in the abdominal cavity, is particularly preferable. By transplanting tooth germ cells into such a site, the growth of the tooth germ cells can be promoted, and formation of tooth germ can be accelerated.

Tooth germ regenerated by the above-described method of the present invention for regenerating tooth germ (which may be either tooth germ tissues obtained by culturing tooth germ cells in the presence of a physiologically active substance, or tooth germ tissues obtained by transplanting the above tooth germ tissues into a transplanted animal and allowing them to further regenerate in the body of the transplanted animal) is transplanted into the jawbone of a patient who has lost a tooth or has had a tooth damaged, so as to treat the dental patient. That is to say, a method for treating a dental patient using tooth germ obtained by the method of the present invention for regenerating tooth germ is also included in the scope of the present invention. Even after tooth germ has been transplanted into the jawbone of a dental patient, the tooth germ is allowed to continuously grow, so as to form a tooth. Otherwise, a tooth root may be formed from tooth germ outside the body of a patient. The tooth root may be transplanted into the jawbone of the patient, and a tooth crown may be then formed by the conventional dental methods.

The present invention will be further specifically described in the following examples. However, the present invention is not limited by these examples.

### EXAMPLES

The present invention will be described in the following examples. However, the present invention is not limited to the examples.

### Reference Example 1

### Isolation of Tooth Germ and Tooth Germ Cells

Using a bone chisel, the tooth germ of the third molar tooth was aseptically extirpated from the lower jawbone of a fresh swine with an age of 6 months old. From this tooth germ, calcified tissues were eliminated in 30 ml of PBS(−) (phosphate buffered saline) solution containing 2 ml of penicillin/streptomycin, and the remaining portion was then transferred into DMEM medium (produced by adding 10% fetal bovine serum, 2% penicillin/streptomycin, and 2% glutamax to Dulbecco's Modified Eagle medium). Subsequently, the resultant was subjected to an enzyme treatment with 2 mg/ml collagenase at 37° C. for 50 minutes. Thereafter, the resultant was centrifuged (1,500 rpm, 8 minutes) and then subjected to a filtration treatment with a 70-μm cell strainer, so as to obtain cells. The obtained cells were cultured in DMEM medium (produced by adding 10% fetal bovine serum and 2% penicillin/streptomycin (100 units/100 μg/ml) to DMEM medium) for 3 weeks (37° C., 5% CO₂). After the cells had become confluent, they were subjected to subculture. When the thus subcultured cells became 70% confluent, they were separated with 0.25% trypsin-EDTA. The obtained cells were used for the subsequent analysis.

### Example 1

### Comparison of Difference in Cell Growth Ability and in Differentiation Induction Ability Between Addition and Not-Addition of Physiologically Active Substance

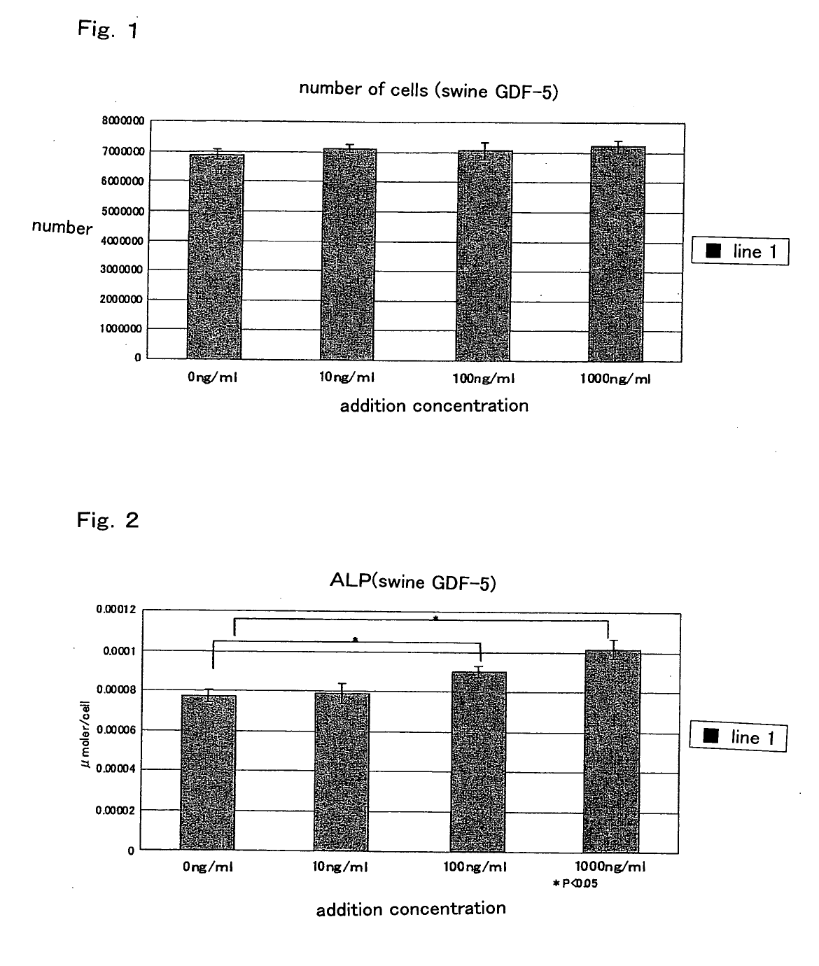

The cells obtained in Reference example 1 were inoculated into a 6-well plate. When the cells became 70% confluent, the serum was eliminated. Thereafter, the cells were washed with PBS(−), and they were then cultured in a serum free medium, to which GDF-5 had been added. The concentration of the added substance was set at 0 ng/ml, 10 ng/ml, 100 ng/ml, and 1,000 ng/ml. Five days after the addition, the number of cells was counted by WST-8Kit, and alkaline phosphatase activity was then measured (by the method of Lowry). The results are shown in FIGS. 1 and 2.

From the results shown in FIG. 1, it is found that the number of cells, to which GDF-5 had been added, was greater than the number of cells, to which GDF-5 had not been added. In addition, alkaline phosphatase activity acting as an indicator of cells that form hard tissues was significantly increased. These results show that the cells, to which GDF-5 was added and which were then cultured, have a high growth ability and a high hard tissue-forming ability. Thus, it was revealed that addition of GDF-5 is advantageous for regeneration of tooth germ.

### Example 2

### Regeneration of Tooth Germ Using Addition of Physiologically Active Substance (In Vivo Evaluation)

Using a bone chisel, the tooth germ of the third molar tooth was aseptically extirpated from the lower jawbone of a fresh swine with an age of 6 months old. From this tooth germ, calcified tissues were eliminated in 30 ml of PBS(−) (phosphate buffered saline) solution containing 2 ml of penicillin/streptomycin, and the remaining portion was then transferred into DMEM medium (produced by adding 10% fetal bovine serum, 2% penicillin/streptomycin, and 2% glutamax to a DMEM medium). Subsequently, the resultant was subjected to an enzyme treatment with 2 mg/ml collagenase at 37° C. for 50 minutes. Thereafter, the resultant was centrifuged (1,500 rpm, 8 minutes) and then subjected to a filtration treatment with a 70-μm cell strainer, so as to obtain cells.

The number of the cells obtained by the aforementioned procedures was counted under a polarization microscope using a hemacytometer. Thereafter, using a medium to which 200 ng/ml GDF-5 had been added, a cell suspension (1×10⁸ cells) was prepared. Using a micropipette in order not to leak the cells, 100 μl each of this cell suspension was inoculated into PGA carrier (PGA mesh; a mesh with a diameter of 11 mm and a thickness of 2 mm was produced, and it was sterilized by immersion in 70% ethanol for 4 hours and then washed with PBS(−)), which was placed in a 48-well plate. Thereafter, the plate was left at rest in an incubator at 37° C. for 1 hour, and it was then used for transplantation.

The abdominal skin of a nude rat F344 with an age of 5 or 6 weeks old was incised, and its greater momentum was pulled out. The carrier in which the cells had been inoculated was wrapped with the greater momentum, and it was then sutured. Thereafter, the muscle coat and the skin were also sutured.

15 weeks after the transplantation, the rat was sacrificed, and the sample was collected. The extirpated sample was fixed with a 10% formalin solution, and it was then embedded in paraffin according to common methods, so as to produce a continuous tissue section. Thereafter, the section was stained with hematoxylin and eosin, and thus, it was observed in a histological manner.

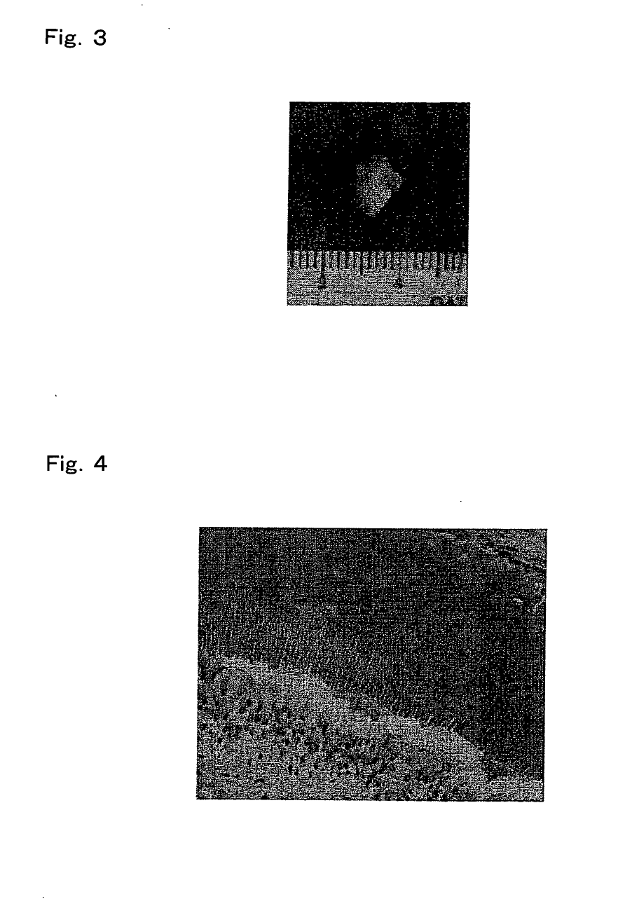

The transplanted product that had been extirpated 15 weeks after the transplantation consisted of tissues containing a hard tissue-like calcified product with a size of approximately 7×7 mm (FIG. 3). It was confirmed that these tissues are much greater than the previously reported regenerated tooth germ tissues that had been extirpated 25 weeks after the transplantation (2×2 mm; J. Dent. Res. 81(10) 2002).

Moreover, as a result of the observation of the tissues stained with hematoxylin and eosin, root dentin-like tissues having a clear dental tubule structure attended with an epithelial sheath and a cementum-like construct were observed in the above tissues. To date, such significant regeneration of a dentin has never been observed in the tooth root formation process. It is considered that addition of a physiological substance to tooth germ cells promotes the growth of a dentin, and that it also promotes formation of a tooth root (FIG. 4).

### Example 3

### Comparison Of Difference in Hard Tissue Formation Ability Between Addition and Not-Addition of FGF-4

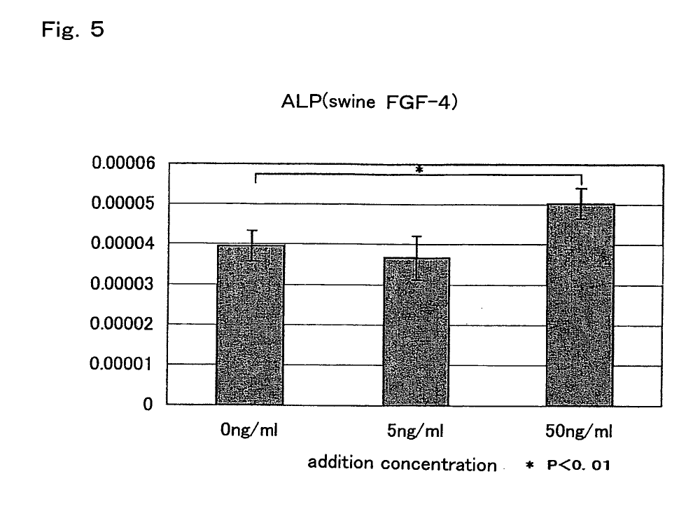

The cells obtained in the same manner as in Reference Example 1 were inoculated into a 12-well plate, and they were then cultured in a medium (produced by adding 10% fetal bovine serum and 1% penicillin/streptomycin to a Dulbecco's Modified Eagle medium), to which FGF-4 had been added. The concentration of the added FGF-4 was set at 0 ng/ml, 5 ng/ml, and 50 ng/ml. Four days after the addition, alkaline phosphatase activity was measured (by the method of Lowry). The results are shown in FIG. 5.

From the results shown in FIG. 5, it is found that alkaline phosphatase activity acting as an indicator of cells that form hard tissues was significantly increased in the cells to which 50 ng/ml FGF-4 had been added, when compared with the case of not adding FGF-4. These results show that the cells which were cultured with addition of FGF-4 have a high hard tissue-forming ability, and it has been found that addition of FGF-4 is advantageous for regeneration of tooth germ.

### INDUSTRIAL APPLICABILITY

Using the method of the present invention, a tooth germ structure such as dentin, dental papilla, or enamel pulp is formed in the body of a transplanted animal or on a medium, or a cell mass is induced to differentiate such that it forms a tooth germ structure. Thereafter, such a tooth germ structure or cell mass, together with a carrier, is transplanted into the jawbone of a dental patient, so as to regenerate a tooth root or tooth. As a result, this becomes a treatment that is extremely effective for recovery of the chewing ability of a patient who has lost a tooth. In addition, such a regenerated tooth enables esthetic recovery, and it greatly contributes to the improvement of the quality of life (QOL) of patients.

1. A method for regenerating tooth germ, which comprises culturing tooth germ cells in the presence of a physiologically active substance.

2. The method for regenerating tooth germ according to claim 1, which comprises culturing tooth germ cells on a carrier in the presence of a physiologically active substance.

3. A method for regenerating tooth germ, which comprises: culturing tooth germ cells in the presence of a physiologically active substance; transplanting the cultured tooth germ cells into a transplanted animal; and regenerating tooth germ in the body of said transplanted animal.

4. A method for regenerating tooth germ, which comprises:
culturing tooth germ cells on a carrier in the presence of a physiologically active substance; transplanting the cultured tooth germ cells together with said carrier into a transplanted animal; and regenerating tooth germ in the body of said transplanted animal.

5. The method for regenerating tooth germ according to claim 1, wherein odontoblasts, ameloblasts, pulp or dental papilla cells, tooth sac cells, or precursor cells thereof, are used as tooth germ cells.

6. The method for regenerating tooth germ according to claim 1, wherein a growth factor is used as a physiologically active substance.

7. The method for regenerating tooth germ according to claim 1, wherein firoblast growth factor (FGF), transforming growth factor (TGF), or a factor belonging to these families, is used as a physiologically active substance.

8. The method for regenerating tooth germ according to any claim 1, wherein GDF-5 or FGF-4 is used as a physiologically active substance.

9. A tooth germ regenerated by the method according to claim 1.

10. A method for treating patients with dental diseases, which comprises transplanting the tooth germ regenerated by the method according to claim 1, into the jawbone of a patient who has lost his or her own tooth germ or has had his or her own tooth damaged.

11. An implant used for live organ transplant, which is obtained by culturing tooth germ cells in the presence of a physiologically active substance.

12. The implant according to claim 11, which is obtained by culturing tooth germ cells on a carrier in the presence of a physiologically active substance.

13. An implant used for live organ transplant, which is obtained by: culturing tooth germ cells in the presence of a physiologically active substance; transplanting the cultured tooth germ cells into a transplanted animal; and regenerating tooth germ in the body of said transplanted animal.

14. An implant used for live organ transplant, which is obtained by: culturing tooth germ cells on a carrier in the presence of a physiologically active substance; transplanting the cultured tooth germ cells together with said carrier into a transplanted animal; and regenerating tooth germ in the body of said transplanted animal.

15. The implant according to claim 11, wherein odontoblasts, ameloblasts, pulp or dental papilla cells, tooth sac cells, or precursor cells thereof, are used as tooth germ cells.

16. The implant according to claim 11, wherein a growth factor is used as a physiologically active substance.

17. The implant according to claim 11, wherein fibroblast growth factor (FGF), transforming growth factor (TGF), or a factor belonging to these families, is used as a physiologically active substance.

18. The implant according to claim 11, wherein GDF-5 or FGF-4 is used as a physiologically active substance.
